(19) United States
(12) Patent Application Publication
(10) Pub. No.: US 2024/0390421 A1
(43) Pub. Date: Nov. 28, 2024

(54) METHODS OF AMPLIFYING TUMOR-REACTIVE IMMUNE POPULATIONS USING ORGANOIDS

(71) Applicant: The Board of Trustees of the Leland Stanford Junior University, Stanford, CA (US)

(72) Inventors: Calvin J. Kuo, Palo Alto, CA (US); Caitlin Grzeskowiak, Redwood City, CA (US)

(21) Appl. No.: 18/691,410

(22) PCT Filed: Sep. 14, 2022
(86) PCT No.: PCT/US2022/043452

§ 371 Date: Mar. 12, 2024

Related U.S. Application Data
(60) Provisional application No. 63/244,422, filed on Sep. 15, 2021.

(51) Int. Cl. A61K 35/17; A61K 39/00; A61P 35/00; C12N 5/0783

(52) U.S. Cl. CPC A61K 35/17; A61K 39/4611; A61P 35/00; C12N 5/0636; C12N 2501/2302; C12N 2501/515; C12N 2501/599; C12N 2502/11; C12N 2502/30; C12N 2513/00

(57) ABSTRACT

A method is provided for treating cancer in an individual, the method comprising culturing patient derived tumor organoids (PDO) with cognate immune cells with or without the presence of one or more direct or indirect T cell activating agents; expanding T cells following activation; and administering the activated T cells to the individual.

### CROSS-REFERENCE TO RELATED APPLICATIONS

Pursuant to 35 U.S.C. § 119 (e), this application claims priority to the filing date of U.S. Provisional Patent Application Ser. No. 63/244,422 filed Sep. 15, 2021, the disclosure of which application is herein incorporated by reference.

### STATEMENT REGARDING FEDERALLY SPONSORED RESEARCH

This invention was made with Government support under contract CA217851 awarded by the National Cancer Institute. The Government has certain rights in the invention.

### BACKGROUND

Cancer treatment paradigms now successfully exploit anti-tumor immunity. In contrast to robust immune reactions to infectious pathogens, tumor-infiltrating lymphocytes (TILs) and other tumor-resident immune cells can be functionally impaired and dysregulated, a condition termed “T cell exhaustion”, which can be exemplified by expression of programmed cell death-1 (PD-1) and other markers such as LAG-3, TIM-3 and TIGIT, which also function as inhibitory immune checkpoints. Melanoma and diverse other cancers have been treated by immune checkpoint blockade (ICB), antagonizing immune evasion engendered by PD-1 and CTLA-4.

In parallel, adoptive cell transfer (ACT) approaches for solid tumors utilize infusion of tumor-reactive T cells, with T cell receptor (TCR) recognition of MHC-bound peptide tumor antigens, although adoptive transfer of CAR-T cells with chimeric antigen TCRs has not yet demonstrated significant success in solid tumors. In contrast, bulk TIL approaches where TILs are extracted from tumors, expanded ex vivo and reinfused autologously into patients, have exhibited encouraging activity in treatment-naïve and/or refractory melanoma including anti-PD-1-resistant patients and cervical cancer. However, current bulk TIL therapy does not selectively enrich for tumor-reactive TILs, a manipulation that could enhance efficacy. Parallel efforts express tumor-reactive TCRs (TCR TILs) or enrich tumor-reactive TILs with increased neoantigen recognition.

The quest for improved immunotherapies would be greatly accelerated by appropriate in vitro systems. However, in vitro cancer modeling with both tumor epithelium and immune stroma has been a formidable challenge with the need to include natively infiltrating TILs, and non-lymphocyte components such as macrophages and NK cells. Immune cells from blood or patient tumors have only been reconstituted with conventional non-syngeneic 2D cancer cell lines as traditional monolayers or spheroids. Recent advances allow human tumors to be grown as 3D “organoids” using a submerged Matrigel method and exogenous growth factors, but such organoids are exclusively comprised of tumor epithelium, do not include tumor stroma unless reconstituted with cancer-associated fibroblasts (CAFs) and do not contain immune cells.

Peripheral blood lymphocytes (PBL) have been used to derive T cell lines that are reactive against primary tumor organoid cultures. Alternatively, murine macrophages and other human immune cell types have been grown in short-term culture with tumor cells, or in custom microfluidic devices as human tumor suspension-derived microspheroids. However, these approaches typically require artificial reconstitution between tumor and immune cells, do not exhibit the full diversity of immune cells (i.e. T, B, macrophage, NK) within the tumor microenvironment (TME) and often do not demonstrate anti-tumor immunity. In vivo immunotherapy models have been hampered by the need to grow human tumors in immunodeficient mouse hosts, which thus lack the very immune component under study. Alternatively, reconstitution of immunodeficient mice with human immune cells can be performed, albeit in an incomplete and/or non-syngeneic manner.

The present disclosure provides methods for the generation of 3-dimensional air-liquid interface organoid systems that culture tumors en bloc with their endogenous immune cells and allow for the expansion of the immune cells present.

### SUMMARY

Culture systems and methods are provided for the generation and expansion of tumor-specific immune cells. Patient derived tumor organoids (PDO) are cultured with cognate immune cells, providing a bioreactor for the functional enrichment of tumor reactive T cells. In some embodiments, methods are provided for the culture of tumor reactive immune cells activated by in vitro immune checkpoint inhibitor (ICI) treatment and culture with the PDO, which activated cells may be further expanded to be used as an immunotherapeutic agent, e.g. for treatment of cancer. The cultures can also provide for methods to identify T cell receptor clonotypes (TCR) within the tumor microenvironment.

Tissue samples for the generation of PDOs can be collected from tumor biopsies. Cancers that produce tumors used for biopsies for the methods of the invention, include without limitation, solid tumors, for example clear cell renal cell carcinoma, ampullary carcinoma, cutaneous SCC, melanoma, lung adenocarcinoma, non-small lung cell cancer, gastrointestinal cancer, pancreatic cancer, colorectal cancer, hepatocellular carcinoma, cholangiocarcinoma, combined hepatocellular cholangiocarcinoma, Barrett's oesophagus cancer, intestinal carcinoma, prostate cancer, bladder cancer, breast cancer, glioblastoma, cutaneous squamous cell carcinoma, etc.

The methods described herein utilize liquid-air interface (ALI) organoid in vitro cultures derived from tissue from a tumor biopsy, where cultures comprise tumor cells and immune stroma cultured from tumor biopsies. The tissue is cultured on a medium that supports maintenance and activity of both tumor and immune cells. Air-liquid interface (ALI) organoids of the disclosure may comprise epithelial and stromal components from the tumor tissue used to initiate the culture. The ALI method allows culturing epithelium and stroma together as a cohesive 3-dimensional unit that recapitulates the function and the micro-anatomy of the organ of origin, and includes endogenous immune cells. In ALI, adequate oxygenation is achieved by culturing microscopic fragments of tissue embedded in a collagen matrix within a trans-well (“inner dish”) in which direct air exposure is obtained from the top; whilst contact with tissue culture media contained in an “outer dish”; is obtained from the bottom via the trans-well permeable membrane.

The cultures comprise immune cells, particularly T cells. T cell subsets of interest include, without limitation, CART cells, naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells (T_{Reg}), e.g. T_{R}1, natural T_{Reg}, inducible T_{Reg}; memory T cells, e.g., central memory T cells, stem cell memory T cells (T_{SCM}), effector memory T cells, NK T cells, γδ T cells; etc. In some embodiments, the cells comprise a complex mixture of immune cells, e.g., tumor infiltrating lymphocytes (TILs) isolated from an individual in need of treatment.

Cultures can comprise exogenous agents that are added to activate T cells present in the culture. Agents that activate T cells and can be added to the culture may include, for example, immune checkpoint inhibitors, e.g. agents such as antibodies that inhibit the activity of CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T cell Immunoglobulin domain and Mucin domain 3), VISTA (V-domain Ig suppressor of T cell activation), IL-2R (interleukin-2 receptor), T cell immunoreceptor with immunoglobulin and ITIM domain (TGIT), etc. In some embodiments, a combination of agents that activate T cells are added to cultures. Combinations of agents may include a combination of two or more of the any of the agents listed above. Activation strategies can include protocols to reverse T cell exhaustion, e.g. pulsatile stimulation, addition of kinase inhibitors such as dasatinib, and the like. T cells may be indirectly activated and expanded by antecedent blockade of macrophage phagocytosis inhibitory pathways, i.e. “don't eat me” signals. Thus antibodies blocking the interaction of macrophage phagocytosis inhibitory molecules such as CD47 and SIRPα can enhance phagocytosis of tumor cells, increase antigen presentation to T cells and thus indirectly activate and expand T cells.

When PDOs are cultured with agents that activate T cells, either directly or indirectly, they can be cultured for any period of time deemed necessary to activate T cells. Culturing time with agents that activate T cells may be for up to 2 days, up to 3 days, up to 4 days, up to 5 days, up to 6 days, up to 7 days, up to 8 days, up to 9 days, up to 10 days or more than 10 days. Following culturing with one or more T cell activating agents, T cell activation can be assessed. Activated T cells can be identified and optionally quantitated based on a number of criteria. The criteria include, without limitation, expression of CD3, CD25, CD69, CD137, CD107A, Granzyme B (GZMB), Perforin 1 (PRF1), etc. Activated T cells can be isolated based on expression of these activation markers. Non-activated PDO cultures (i.e. cultures that were not treated with a T cell activation agent) may be used as a control.

Following activation, the T cells can be further expanded. In some embodiments, expansion of T cells occur through the use of a rapid expansion protocol. In some embodiments, the rapid expansion protocol comprises culturing T cells in a non-ALI culture, for example in culture comprising IL-2, an anti-CD3 antibody, and irradiated allogenic peripheral blood mononuclear cell (PBMC) feeders. In some embodiments, the anti-CD3 antibody is the monoclonal OKT3 antibody. In some embodiments, T cells are expanded using the rapid expansion program for up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days or greater. Once expanded, an effective dose of T cells can then be administered to a patient, including without limitation the patient from which the PDOs were derived from, where the effective dose may be at least about 10² cells, at least about 10³ cells, at least about 10⁴ cells, at least about 10⁵ cells, at least about 10⁶ cells, at least about 10⁷ cells, or more, which may be delivered systemically, by intratumoral injection, etc.

In some embodiments, expanded T cells are assayed for functional activity. Assays for functional activity include, without limitation, T cell cytotoxicity assays, IL-2 response, etc. as known in the art. Alternatively the T cells are assessed for the presence of markers indicative of activation, e.g. expression of CD3, CD25, CD69, CD137, CD107A, Granzyme B (GZMB), Perforin 1 (PRF1); etc. T cells can also be selected for an activated phenotype prior to administration.

In some embodiments, a method is provided for treating cancer in an individual, the method comprising culturing patient derived tumor organoids (PDO) with cognate immune cells in the presence of one or more T cell activating agents; expanding T cells following activation; and administering the activated T cells to the individual. In some embodiments, the T cells are autologous, in others allogeneic T cells are used. In some embodiments, the T cells are tumor-infiltrating T cells. In some embodiments, the T cells are selected for activation prior to administration.

### BRIEF DESCRIPTION OF THE DRAWINGS

FIG. 1. Organoid-based single/multiple checkpoint blockade enrichment of tumor-reactive TILs for adoptive immunotherapy. A. ALI organoids from mouse and human tumors are untreated or treated with immune checkpoint inhibitors (targeting T cells, macrophages or other populations) to enrich tumor-reactive TILs. B. Ex vivo CD3+ TIL expansion. C. Expanded TILs are functionally evaluated for anti-tumor activity in vitro and in vivo.

FIG. 2. Expanded TILs after PD-1 antibody treatment induce apoptosis of tumor cells through MHC-restricted manner. Patient derived organoid from renal cell carcinoma were treated by control versus PD-1 antibody in ALI organoids for 7 days. ALI organoids were extracted by dissolving gels with collagenase after the treatment, and placed into T cell expansion plates (G-REX, Wilson Wolf Manufacturing) with T cell culture media supplemented by CD3/28/2antibody and IL-2 (6000 IU/ml). To assess T cell killing of autologous tumor cells, 50,000 cells of CD8+ T cells purified by FACS sorting were co-cultured with tumor organoids derived from the same sample but without immune cells, in 5:1 effector: target ratio, compared with several wells including only tumor or T cells in 96 well plate for 72 hours at 37° C. In some wells, anti-MHC-class I antibody (Biolegend, clone W6/32) was added to evaluate whether the tumor cells were recognized through MHC-restricted manner. To detect apoptosis, Cell Event-3/7 Green Detection Reagent (Invitrogen) was added to all of the wells and incubated for 30 minutes following the manufacturer's protocol. Fluorescence intensity was measured by plate reader. Mean fluorescence intensity of each condition is shown in bar graph. Data are expressed as the mean with standard deviation. As opposed to TILs expanded from control antibody treated tumor organoids, TILs expanded from anti-PD1-treated tumor organoids increased subsequent in vitro killing of tumor cells. This effect was reversed by MHC-block with anti-MHC class I antibody.

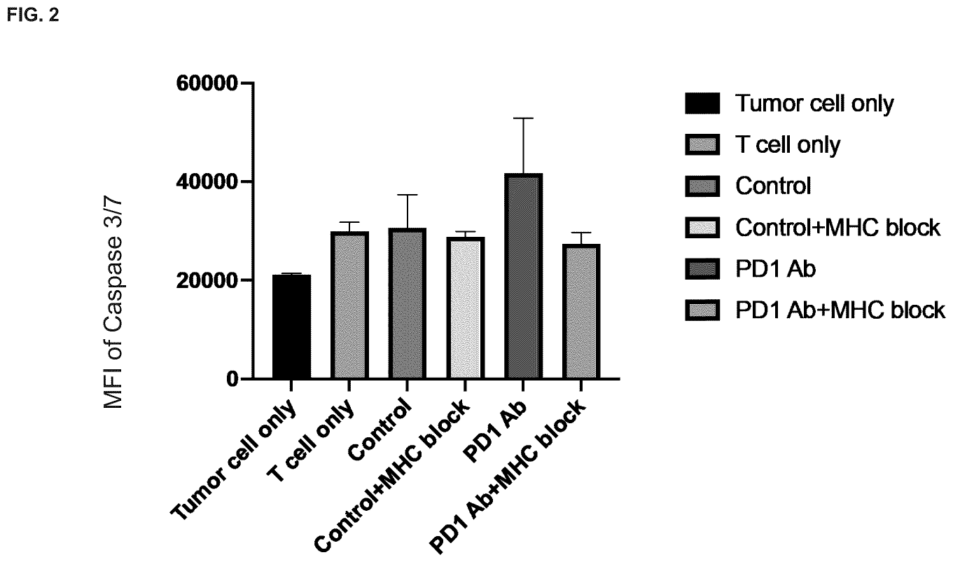

### DETAILED DESCRIPTION

In vitro cancer modeling and bioreactor design presents a formidable challenge, as tumor development and progression rely on not only a multiplicity of genetic and molecular alterations, but also physical and spatial factors within a 3-dimensional microenvironment composed of numerous cell types. While recent in vitro models have attempted to integrate tumor architecture by culturing primary human tumors, these models do not recapitulate higher-order phenomena in tumor progression involving stromal and/or immune interactions. Here we present a patient derived organoid (PDO) culture system that accurately recapitulates complex tumor architecture and histology including tumor parenchymal, stromal, and immune compartments without the need for grafting in a non-human host. Using a single 3-dimensional air-liquid interface methodology, a large number of unique PDO cultures from wide variety of human neoplasms are generated. These PDOs are used as a bioreactors to culture and expand cognate immune cells, i.e. T-cells such as TILs, to produce isolated activated immune cells that can be used as a cancer immunotherapeutic.

In the description that follows, a number of terms conventionally used in the field of cell culture are utilized extensively. In order to provide a clear and consistent understanding of the specification and claims, and the scope to be given to such terms, the following definitions are provided.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

The term “culture system” is used herein to refer to the culture conditions in which the subject explants are grown that promote prolonged tissue expansion with proliferation, multilineage differentiation and recapitulation of cellular and tissue ultrastructure. A culture system also refers to a non-ALI culture in which cells of interest can be expanded, e.g. on feeder layer cells.

Culture conditions of interest provide an environment permissive for differentiation, in which the complex cell system from an explant cells will proliferate, differentiate, or mature in vitro. Such conditions may also be referred to as “differentiative conditions”. Features of the environment include the medium in which the cells are cultured, any growth factors or differentiation-inducing factors that may be present, and a supporting structure (such as a substrate on a solid surface) if present.

“Gel substrate”, as used herein has the conventional meaning of a semi-solid extracellular matrix. Gel described herein includes without limitations, collagen gel, matrigel, extracellular matrix proteins, fibronectin, collagen in various combinations with one or more of laminin, entactin (nidogen), fibronectin, and heparin sulfate; and human placental extracellular matrix.

An “air-liquid interface” is the interface to which the tumor cells are exposed to, in the cultures described herein. The primary tissue may be mixed with a gel solution which is then poured over a layer of gel formed in a container with a lower semi-permeable support, e.g. a membrane. This container is placed in an outer container that contains the medium such that the gel containing the tissue in not submerged in the medium. The primary tissue is exposed to air from the top and to liquid medium from the bottom, see for example U.S. Pat. No. 9,464,275,herein specifically incorporated by reference.

By “container” is meant a glass, plastic, or metal vessel that can provide an aseptic environment for culturing cells.

The term “explant” is used herein to mean a piece of tumor tissue and the cells thereof originating from the tumor tissue that is cultured in vitro, for example according to the methods of the invention. The tissue from which the explant is derived is obtained from an individual, i.e. a cancer patient. Methods of interest include patient-specific analysis of anti-tumor immune responses.

The term “organoid” is used herein to mean a 3-dimensional growth of tumor tissue in culture that retains characteristics of the tumor in vivo, e.g. recapitulation of cellular and tissue ultrastructure, immune cell interactions, etc. Organoids for use in the methods disclosed herein are generally cultured from a tumor biopsy section. Organoids may be generated using any method known in the art, depending on the application. Methods of organoid culture that find use in the present disclosure, include without limitation, a submerged method, an air liquid interface method, a suspension culture method, a droplet and bioreactor method, etc.

Methods are provided for the culture of small amounts of clinical specimens. Samples of interest include human tissue, particularly cancer and other lesions, e.g. solid tumor microbiopsy samples such as needle or fine needle aspirate. Samples may be taken at a single timepoint, or may be taken at multiple timepoints. Samples may be as small as 10⁷ cells, 10⁶ cells, 10⁵ cells, or less; e.g. a tumor biopsy section of from about 0.1 mm², about 1 mm², about 10 mm², etc.

The air-liquid interface (ALI) method allows the propagation of organoids both with epithelial and stromal components of tumors. The ALI method utilizes Boyden chambers (cell culture inserts) used for cell migration assays. Cells are embedded in ECM gels in an upper surface of the cell culture inserts with a porous membrane underneath and cells are directly exposed to oxygen, which substantially increases the oxygen supply to the cells as compared to an epithelial-only submerged organoid method. Cells obtain nutrients and growth factors from the medium placed in the outer dish through diffusion across the porous membrane on the lower surface. The distinct advantage of the ALI method is that it not only includes stromal cells but can also retain the tumor microenvironment for an extended period of time. Methods of using the ALI method to culture PDOs is known within, for instance, as disclosed by Neal et al. Cell. 2018 Dec. 13; 175 (7): 1972-1988.e16 which is incorporated herein in its entirety by reference

In alternative organoid cultures, e.g. the droplet and bioreactor method, tissue is embedded into droplets of BME and then transferred into spinning bioreactors. The continuous agitation in this method provides improved absorption of nutrients and oxygen. Very recently, glioblastoma organoids were prepared using this similar agitation method, but without mitogens and BME and with a defined culture medium. Interestingly, glioblastoma organoids generated using this method retained histological, genetic features and partial preservation of the microvasculature, as well as immune cells of the original tumor.

Additionally, organoids may be formed in suspension, or formed in solid extracellular matrix gels and then transferred into suspension culture, or vice versa.

As used herein, the term “immune cell” includes cells that are of hematopoietic origin and that play a role in the immune response. Immune cells include lymphocytes, such as B cells and T cells; natural killer cells; dendritic cells; myeloid cells, such as monocytes, macrophages, eosinophils, mast cells, basophils, and granulocytes.

The term “T cells” refers to mammalian immune effector cells that may be characterized by expression of CD3 and/or T cell antigen receptor, which cells can be engineered to express a CD25 variant or IL-13Rα2 protein. In some embodiments, the T cells are selected from naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, T stem cell memory cells (T_{SCM}). effector memory T cells, NKT cells, γδ T cells.

In some embodiments, the immune cells comprise a complex mixture of immune cells, e.g., tumor infiltrating lymphocytes (TILs) isolated from an individual in need of treatment. See, for example, Yang and Rosenberg (2016) Adv Immunol. 130: 279-94, “Adoptive T Cell Therapy for Cancer; Feldman et al (2015) Semin Oncol. 42 (4): 626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344 (6184) 641-645, “Cancer immunotherapy based on mutation-specific CD4+ T cells in a patient with epithelial cancer”.

T effector cells, for the purposes of the disclosure, can include autologous or allogeneic immune cells having cytolytic activity against a target cell, including without limitation tumor cells. The effector cells may have cytolytic activity that does not require recognition through the T cell antigen receptor. Cells of particular interest include cells of the T cell and/or natural killer cell (NK) lineage. The cells are optionally separated from non-desired cells prior to culture, prior to administration, or both. Cell-mediated cytolysis of target cells by immunological effector cells is mediated by the local directed exocytosis of cytoplasmic granules that penetrate the cell membrane of the bound target cell.

Natural killer (NK) cells are cytotoxic cells belonging to a cell class responsible for cellular cytotoxicity without prior sensitization. For example, IL-2-activated NK cells, the major effector population in lymphokine-activated killer (LAK) cells, are potent mediators of the lysis of autologous and allogeneic leukemic cells in vitro. LAK cells are non-B, non-T cells that are capable of recognizing cancer cells in a non-MHC-restricted fashion. LAK cells, which can be generated from either the normal or tumor-bearing host, appeared to represent a primitive immunosurveillance system capable of recognizing and destroying altered cells. NK cells often do not react with patient tumor cells unless they are activated by interferon, IL-2, or unless suppressor monocytes are removed from the effector cell population, and thus can benefit from engineering to express a high affinity CD25 protein. IL-2 induces proliferation of T lymphocytes and NK cells and the production of IFN-gamma; it also results in the induction of LAK cells against previously NK-resistant cell preparations and cell lines. LAK activity can be generated from human and murine T cells following engineering, and incubation with IL-2. LAK cells have been utilized in vivo both in animals and in human beings for the treatment of melanoma, renal cell carcinoma, non-Hodgkin's lymphoma, and lung and colorectal cancers.

Cytotoxic T lymphocytes (CTL) reactive to autologous tumor cells are specific effector cells for adoptive immunotherapy and are of interest for engineering according to the methods described herein. Induction and expansion of CTL is antigen-specific and MHC restricted.

Cytokine-induced killer (CIK) cells are highly efficient cytotoxic effector cells obtained by culturing peripheral blood lymphocytes (PBLs) in the presence of IFN-γ, IL-2 (or IL-12), and monoclonal antibody (MAb) against CD3, and optionally IL-1α. Cells may be cultured for at least about 1 week, at least about 2 week, at least about 3 weeks, or more, and usually not more than about 8 weeks in culture. The absolute number of CIK effector cells usually increases at least about 100-fold in such culture conditions, and may increase by at least about 500-fold, at least about 1000-fold, or more. CIK cells possess a higher level of cytotoxic activity and a higher proliferation rate than LAK cells. The phenotype of the cells with the greatest cytotoxicity expresses both the T-cell marker CD3 and the NK cell marker CD56. The dominant cell phenotype in CIK cell cultures expressed the alpha-, beta-T-cell receptor (TCR-α/β). In comparison to NK cells, the cytotoxicity mediated by CD3⁺CD56⁺ cells is also non-MHC restricted in the absence of activation, but it is non-ADCC dependent, since these double-positive cells do not express CD16. Morphologically, these cells cannot be distinguished from NK cells.

The phrase “mammalian cells” means cells originating from mammalian tissue. Typically, in the methods of the invention, pieces of tissue are obtained surgically, e.g. biopsy, needle biopsy, etc. and minced to a size less than about 1 mm³, and may be less than about 0.5 mm³, or less than about 0.1 mm³. “Mammalian” used herein includes human, equine, bovine, porcine, canine, feline, rodent, e.g. mice, rats, hamster, primate, etc. “Mammalian tissue cells” and “primary cells” have been used interchangeably.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue. Cancer immunotherapy is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates).

Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and self-antigens, e.g. her-2/neu; carcinoembryonic antigen, muc-1, and the like.

Active immunotherapy, which may be referred to as immune-oncology, directs the immune system to attack tumor cells by targeting TAAs. Passive immunotherapies enhance existing anti-tumor responses and include the use of monoclonal antibodies, lymphocytes and cytokines.

Immune Responsiveness Modulators. Immune checkpoint proteins are immune inhibitory molecules that act to decrease immune responsiveness toward a target cell, particularly against a tumor cell in the methods of the invention. Endogenous responses to tumors by T cells can be dysregulated by tumor cells activating immune checkpoints (immune inhibitory proteins) and inhibiting co-stimulatory receptors (immune activating proteins). The class of therapeutic agents referred to in the art as “immune checkpoint inhibitors” reverses the inhibition of immune responses through administering antagonists of inhibitory signals. Other immunotherapies administer agonists of immune costimulatory molecules to increase responsiveness. Antibodies blocking the interaction of CD47 and SIRPα can enhance phagocytosis of tumor cells via action on macrophages or other immune cell types.

Immune-checkpoint receptors that have been most actively studied in the context of clinical cancer immunotherapy, cytotoxic T-lymphocyte-associated antigen 4 (CTLA4; also known as CD152) and programmed cell death protein 1 (PD1; also known as CD279)—are both inhibitory receptors. The clinical activity of antibodies that block either of these receptors implies that antitumor immunity can be enhanced at multiple levels and that combinatorial strategies can be intelligently designed, guided by mechanistic considerations and preclinical models.

CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity. CTLA4 blockade results in a broad enhancement of immune responses. Two fully humanized CTLA4 antibodies, ipilimumab and tremelimumab, are in clinical testing and use. Clinically the response to immune-checkpoint blockers is slow and, in many patients, delayed up to 6 months after treatment initiation. In some cases, metastatic lesions actually increase in size on computed tomography (CT) or magnetic resonance imaging (MRI) scans before regressing. Anti-CTLA4 antibodies that antagonize this inhibitory immune function are very potent therapeutics but have significant side effects since this enables T cell activity against the self that is usually inhibited through these inhibitory molecules and pathways.

CTLA4 is expressed on regulatory T cells that inhibit T cell activation and expansion and anti-CTLA4 antibodies block their inhibitory immunosuppressive function. As a result, anti-tumor T cells can be/stay activated and expand. One aspect of this effect is the inhibition of the inhibitory signaling pathway but another aspect is the depletion of regulatory T cells that express CTLA4.

Other immune-checkpoint proteins are PD1 and PDL1. Antibodies in current clinical use against these targets include nivolumab and pembrolizumab. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand. Because many tumors are highly infiltrated with T_{Reg} cells, blockade of the PD1 pathway may also enhance antitumor immune responses by diminishing the number and/or suppressive activity of intratumoral T_{Reg} cells.

The two ligands for PD1 are PD1 ligand 1 (PDL1; also known as B7-H1 and CD274) and PDL2 (also known as B7-DC and CD273). The PD1 ligands are commonly upregulated on the tumor cell surface from many different human tumors. On cells from solid tumors, the major PD1 ligand that is expressed is PDL1. PDL1 is expressed on cancer cells and through binding to it's receptor PD1 on T cells it inhibits T cell activation/function. Therefore, PD1 and PDL1 blocking agents can overcome this inhibitory signaling and maintain or restore anti-tumor T cell function.

PDL1 is expressed on cancer cells and through binding to its receptor PD1 on T cells it inhibits T cell activation/function. Therefore, PD1 and PDL1 blocking agents can overcome this inhibitory signaling and maintain or restore anti-tumor T cell function.

Lymphocyte activation gene 3 (LAG3; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors are associated with the inhibition of lymphocyte activity and in some cases the induction of lymphocyte energy. Antibody targeting of these receptors can be used in the methods of the invention.

LAG3 is a CD4 homolog that enhances the function of T_{Reg} cells. LAG3 also inhibits CD8⁺ effector T cell functions independently of its role on T_{Reg} cells. The only known ligand for LAG3 is MHC class Il molecules, which are expressed on tumor-infiltrating macrophages and dendritic cells. LAG3 is one of various immune-checkpoint receptors that are coordinately upregulated on both T_{Reg} cells and anergic T cells, and simultaneous blockade of these receptors can result in enhanced reversal of this anergic state relative to blockade of one receptor alone. In particular, PD1 and LAG3 are commonly co-expressed on anergic or exhausted T cells. Dual blockade of LAG3 and PD1 synergistically reversed anergy among tumor-specific CD8⁺ T cells and virus-specific CD8⁺ T cells in the setting of chronic infection. LAG3 blocking agents can overcome this inhibitory signaling and maintain or restore anti-tumor T cell function.

TIM3 inhibits T helper 1 (TH1) cell responses, and TIM3 antibodies enhance antitumor immunity. TIM3 has also been reported to be co-expressed with PD1 on tumor-specific CD8⁺ T cells. Tim3 blocking agents can overcome this inhibitory signaling and maintain or restore anti-tumor T cell function.

BTLA is an inhibitory receptor on T cells that interacts with TNFRSF14. BTLA^{hi} T cells are inhibited in the presence of its ligand. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions. Dual blockade of BTLA and PD1 enhances antitumor immunity. A2aR, the ligand of which is adenosine, inhibits T cell responses, in part by driving CD4⁺ T cells to express FOXP3 and hence to develop into T_{Reg} cells. Deletion of this receptor results in enhanced and sometimes pathological inflammatory responses to infection. A2aR can be inhibited either by antibodies that block adenosine binding or by adenosine analogues.

The term “immune checkpoint inhibitor” refers to a molecule, compound, or composition that binds to an immune checkpoint protein and blocks its activity and/or inhibits the function of the immune regulatory cell expressing the immune checkpoint protein that it binds (e.g., Treg cells, tumor-associated macrophages, etc.). Immune checkpoint proteins may include, but are not limited to, CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T-cell Immunoglobulin domain and Mucin domain 3), VISTA (V-domain Ig suppressor of T cell activation), and IL-2R (interleukin-2 receptor).

Immune checkpoint inhibitors are well known in the art and are commercially or clinically available. These include but are not limited to antibodies that inhibit immune checkpoint proteins. Illustrative examples of checkpoint inhibitors, referenced by their target immune checkpoint protein, are provided as follows. Immune checkpoint inhibitors comprising a CTLA-4 inhibitor include, but are not limited to, BMS-986218, ADG116, ADG126, ONC-392, XTX101, BMS-986288, botensilimab, quavonlimab, tremelimumab, and ipilimumab (marketed as Yervoy).

Immune checkpoint inhibitors comprising a PD-1 inhibitor include, but are not limited to, nivolumab (Opdivo), pidilizumab (CureTech), AMP-514 (MedImmune), pembrolizumab (Keytruda), AUNP 12 (peptide, Aurigene and Pierre), Zeluvalimab, Pimivalimab, LVGN3616, Sym021, SYN125, sasanlimab, toripalimab, tislelizumab, tebotelimab, zimberelimab, Cemiplimab (Libtayo), and Balstilimab. Immune checkpoint inhibitors comprising a PD-L1 inhibitor include, but are not limited to, IMC-001, envafolimab,, BMS-936559/MDX-1105 (Bristol-Myers Squibb), MPDL3280A (Genentech), MED14736 (MedImmune), MSB0010718C (EMD Sereno), Atezolizumab (Tecentriq), Avelumab (Bavencio), and Durvalumab (Imfinzi).

Immune checkpoint inhibitors comprising a B7-H3 inhibitor include, but are not limited to, MGA271 (Macrogenics). Immune checkpoint inhibitors comprising an LAG3 inhibitor include, but are not limited to, IMP321 (Immuntep), encelimab, and BMS-986016 (Bristol-Myers Squibb). Immune checkpoint inhibitors comprising a KIR inhibitor include, but are not limited to, IPH2101 (lirilumab, Bristol-Myers Squibb). Immune checkpoint inhibitors comprising an OX40 inhibitor include, but are not limited to INCAGN01949, revdofilimab, BGB-a445, BMS 986178, GSK3174998, ivuxolimab, and MEDI-6469 (MedImmune). An immune checkpoint inhibitor targeting IL-2R, for preferentially depleting Treg cells (e.g., FoxP-3+ CD4+ cells), comprises IL-2-toxin fusion proteins, which include, but are not limited to, denileukin diftitox (Ontak; Eisai). Further, phagocytosis immune checkpoint inhibitors can target the interaction of CD47 and SIRPα such as clones B6H12, 5F9, 8B6, and C3.

CD47 is a broadly expressed transmembrane glycoprotein with a single Ig-like domain and five membrane spanning regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells. Structural determinants on SIRPα that mediate CD47 binding are discussed by Lee et al. (2007) J. Immunol. 179: 7741-7750; Hatherley et al. (2008) Mol Cell. 31 (2): 266-77; Hatherley et al. (2007) J. B. C. 282: 14567-75; and the role of SIRPα cis dimerization in CD47 binding is discussed by Lee et al. (2010) J. B. C. 285: 37953-63. In keeping with the role of CD47 to inhibit phagocytosis of normal cells, there is evidence that it is transiently upregulated on hematopoietic stem cells (HSCs) and progenitors just prior to and during their migratory phase, and that the level of CD47 on these cells determines the probability that they are engulfed in vivo.

In some embodiments, the immune checkpoint inhibitor is an anti-CD47 antibody. The desired anti-CD47 antibody is an antibody that specifically binds CD47 (i.e., an anti-CD47antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody.” A suitable anti-CD47antibody that is an “anti-CD47 agent” can be referred to as a “CD47-blocking antibody”. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies (e.g., hu5F9-G4, magrolimab) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments an anti-CD47 antibody comprises a human IgG Fc region, e.g. an IgG1, IgG2a, IgG2b, IgG3, IgG4 constant region. In a preferred embodiment the IgG Fc region is an IgG4 constant region. The IgG4 hinge may be stabilized by the amino acid substitution S241P (see Angal et al. (1993) Mol. Immunol. 30 (1): 105-108, herein specifically incorporated by reference).

MHC Proteins. Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class Il.

MHC context. The function of MHC molecules is to bind peptide fragments derived from pathogens or aberrant proteins derived from transformed cells, and display them on the cell surface for recognition by the appropriate T cells. Thus, T cell receptor recognition can be influenced by the MHC protein that is presenting the antigen. The term MHC context refers to the recognition by a TCR of a given peptide, when it is presented by a specific MHC protein.

Class I HLA/MHC. For class I proteins, the binding domains may include the α1, α2 and optionally α3 domain of a Class I allele, including without limitation HLA-A, HLA-B, HLA-C, H-2K, H-2D, H-2L, which are combined with B2-microglobulin. In certain specific embodiments, the binding domains are HLA-A2 binding domains, e.g. comprising at least the alpha 1 and alpha 2domains of an A2 protein. A large number of alleles have been identified in HLA-A2, including without limitation HLA-A*02:01:01:01 to HLA-A*02:478, which sequences are available at, for example, Robinson et al. (2011), The IMGT/HLA database. Nucleic Acids Research 39 Suppl 1: D1171-6. Among the HLA-A2 allelic variants, HLA-A*02:01 is the most prevalent. Many immune checkpoint inhibitors are antibodies.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also refers to single domain antibodies or nanobodies. The term antibody also includes bivalent or bispecific molecules, diabodies, triabodies, and tetrabodies.

A “single-chain antibody,” “single chain variable fragment,” or “scFv” has an antibody heavy chain variable domain (VH) and a light-chain variable domain (VL) joined together by a flexible peptide linker. The peptide linker is typically 10-25 amino acids in length. Single-chain antibodies retain the antigen-binding properties of natural full-length antibodies, but are smaller than natural intact antibodies or Fab fragments because of the lack of an Fc domain.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363: 446; Desmyter et al. (2015) Curr. Opin. Struct. Biol. 32:1). In the family of “camelids” immunoglobulins devoid of light polypeptide chains are found. “Camelids” include old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody. Nanobodies are smaller than human antibodies, where nanobodies are generally 12-15 kDa, human antibodies are generally 150-160 kDa, Fab fragments are ˜50 kDa and single-chain variable fragments are ˜25 kDa. Nanobodies provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like. Specific nanobodies have been successfully generated using yeast surface display as shown in McMahon et al. (2018) Nature Structural Molecular Biology 25 (3): 289-296 which is specifically incorporated herein by reference.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of interest bind antigens on the surface of target cells in the presence of effector cells (such as natural killer cells or macrophages). Fc receptors on effector cells recognize bound antibodies.

An antibody immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

Tissues for use in the methods disclosed herein include without limitation, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia tissue.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade | cancer, grade II cancer, grade III cancer, malignant cancer and primary carcinomas are included.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence. The present methods allow prediction of whether a patient will be responsive to a therapy of interest.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

### Methods

Culture systems and methods are provided for the generation and expansion of tumor-specific immune cells by organoid culture of solid tumors, including stromal and immune cells associated with the tumors in vivo, to activate and expand T cells, e.g. TILs, specific for the tumor-associated antigens. The PDO cultures can be maintained for up to 5 days, up to 7 days, up to 10 days, up to 15 days, up to 21 days, up to 28 days, or more. In some embodiments, tissue, i.e. primary tissue, is obtained from a solid tumor. The tumor tissue may be from any mammalian species, e.g. human, equine, bovine, porcine, canine, feline, rodent, e.g. mice, rats, hamster, primate, etc.

PDO culture conditions of the disclosure comprise cognate immune cells, e.g. endogenous immune cells present in the biopsy sample; allogeneic T cells; etc. Immune cells that can be cultured with PDOs include, without limitation, T cells, macrophages, B cells, natural killer cells (NK cells), etc., including any of the T cell subsets as discussed herein.

Tumor tissue may be obtained by any convenient method, e.g. by biopsy, e.g. during endoscopy, during surgery, by needle, etc., and is typically obtained as aseptically as possible. Upon removal, tissue is immersed in ice-cold buffered solution, e.g. PBS, Ham's F12, MEM, culture medium, etc. Pieces of tissue may be minced to a size less than about 1 mm³, and may be less than about 0.5 mm³, or less than about 0.1 mm³. The minced tissue is mixed with a gel substrate, e.g. a collagen gel solution, e.g. Cellmatrix type I-A collagen (Nitta Gelatin Inc.); a matrigel solution, etc. Subsequently, the tissue-containing gel substrate is layered over a layer of gel (a “foundation layer”) in a container with a lower semi-permeable support, e.g. a membrane, supporting the foundation gel layer, and the tissue-containing gel substrate is allowed to solidify. This container is placed into an outer container containing a suitable medium, for example HAMs F-12 medium supplemented with fetal calf serum (FCS) at a concentration of from about 1 to about 25%, usually from about 5 to about 20%, etc.

The arrangement described above allows nutrients to travel from the bottom, through the membrane and the foundation gel layer to the gel layer containing the tissue. The level of the medium is maintained such that the top part of the gel, i.e. the gel layer containing the explants, is not submerged in liquid but is exposed to air. Thus the tissue is grown in a gel with an air-liquid interface. A description of an example of an air-liquid interface culture system is provided in Ootani et al. in Nat Med. 2009 June; 15 (6): 701-6, the disclosure of which is incorporated herein in its entirety by reference. The air-liquid interface organoid cultures can be moved into other formats such as multi-wells for screening or in submerged 2D or 3D geometries where the cells are placed underneath the tissue culture medium.

Cultures can comprise exogenous agents that are added to activate T cells present in the culture. In some embodiments, no specific treatment may be added. In some embodiments, agents that activate T cells can be added to the culture and may include, for example, immune checkpoint inhibitors, e.g. agents such as antibodies that inhibit the activity of CTLA4 (Cytotoxic T-Lymphocyte-Associated protein 4, CD152), PD1 (also known as PD-1; Programmed Death 1 receptor), PD-L1, PD-L2, LAG-3 (Lymphocyte Activation Gene-3), OX40, A2AR (Adenosine A2A receptor), B7-H3 (CD276), B7-H4 (VTCN1), BTLA (B and T Lymphocyte Attenuator, CD272), IDO (Indoleamine 2,3-dioxygenase), KIR (Killer-cell Immunoglobulin-like Receptor), TIM 3 (T cell Immunoglobulin domain and Mucin domain 3), VISTA (V-domain Ig suppressor of T cell activation), IL-2R (interleukin-2 receptor), T cell immunoreceptor with immunoglobulin and ITIM domain (TGIT), etc. In some embodiments, a combination of agents that activate T cells are added to cultures. Combinations of agents may include a combination of two or more of the any of the agents listed above. Activation strategies can include protocols to reverse T cell exhaustion, e.g. pulsatile stimulation, addition of kinase inhibitors such as dasatinib, and the like.

When inhibition of CTLA4 is desired, a number of different antibodies that inhibit the activity of CTLA4 may be used. Non-limiting examples of antibodies that inhibit CTLA4 include, without limitation, BMS-986218, ADG116, ADG126, ONC-392, XTX101, BMS-986288, botensilimab, quavonlimab, tremelimumab, and ipilimumab.

When inhibition of PD-1 is desired, a range of different antibodies that inhibit the activity of PD-1 may be used. Non-limiting examples of antibodies that inhibit PD-1 include, but are not limited to, nivolumab (Opdivo), pidilizumab (CureTech), AMP-514 (MedImmune), pembrolizumab (Keytruda), AUNP 12 (peptide, Aurigene and Pierre), Zeluvalimab, Pimivalimab, LVGN3616, Sym021, SYN125, sasanlimab, toripalimab, tislelizumab, tebotelimab, zimberelimab, Cemiplimab (Libtayo), Balstilimab.

When inhibition of PD-L1 is desired, a variety of different antibodies that inhibit the activity of PD-L1 may be used. Non-limiting examples of antibodies that inhibit PD-L1 include, without limitation, IMC-001, envafolimab,, BMS-936559/MDX-1105 (Bristol-Myers Squibb), MPDL3280A (Genentech), MED14736 (MedImmune), MSB0010718C (EMD Sereno), Atezolizumab (Tecentriq), Avelumab (Bavencio), and Durvalumab (Imfinzi).

When inhibition of B7-H3 is desired, a number of different antibodies that inhibit the activity of B7-H3 may be used. Non-limiting examples that inhibit B7-H3 inhibitor include, but are not limited to, MGA271 (Macrogenics).

When inhibition of LAG3 is desired, a number of different antibodies that inhibit the activity of LAG3 may be used. Non-limiting examples that inhibit LAG3 inhibitor include, without limitation, to, IMP321 (Immuntep), encelimab, and BMS-986016 (Bristol-Myers Squibb).

When inhibition of KIR is desired, a number of different antibodies that inhibit the activity of KIR may be used. Non-limiting examples that inhibit KIR inhibitor include, but are not limited to, IPH2101 (lirilumab, Bristol-Myers Squibb).

When inhibition of OX40 is desired, a number of different antibodies that inhibit the activity of OX40 may be used. Non-limiting examples that inhibit OX40 inhibitor include, without limitation, INCAGN01949, revdofilimab, BGB-a445, BMS 986178, GSK3174998, ivuxolimab, and MEDI-6469 (MedImmune).

When inhibition of IL-2R is desired, a number of different antibodies that inhibit the activity of IL-2R may be used. Non-limiting examples that inhibit IL-2R inhibitor include, but are not limited to, denileukin diftitox (Ontak; Eisai). Further, phagocytosis immune checkpoint inhibitors can target the interaction of CD47 and SIRPα such as clones B6H12, 5F9, 8B6, and C3.

In some embodiments, PDOs can be cultured with or without agents that activate T cells. PDOs can be cultured for any period of time deemed necessary to activate T cells. Culturing time with agents that activate T cells may be for up to 2 days, up to 3 days, up to 4 days, up to 5 days, up to 6 days, up to 7 days, up to 8 days, up to 9 days, up to 10 days or more than 10 days. Following culturing with one or more T cell activating agents, T cell activation can be assessed. Activated T cells can be identified and optionally quantitated based on a number of criteria. The criteria include, without limitation, expression of CD3, CD25, CD69, CD137, CD107A, Granzyme B (GZMB), Perforin 1 (PRF1), etc. Activated T cells can be isolated based on expression of these activation markers. Non-activated PDO cultures (i.e. cultures that were not treated with a T cell activation agent) may be used as a control.

Following activation, the T cells associated with the PDOs can be further expanded. In some embodiments, expansion of T cells occurs through the use of a rapid expansion protocol. In some embodiments, the rapid expansion protocol comprises culturing T cells in a non-ALI culture, for example in culture comprising IL-2, an anti-CD3 antibody, and irradiated allogenic peripheral blood mononuclear cell (PBMC) feeders. In some embodiments, the anti-CD3 antibody is the monoclonal OKT3 antibody. In some embodiments, T cells are expanded using the rapid expansion program for up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days or greater. Once expanded, an effective dose of T cells can then be administered to a patient, including without limitation the patient from which the PDOs were derived from, where the effective dose may be at least about 10² cells, at least about 10³ cells, at least about 10⁴ cells, at least about 10⁵ cells, at least about 10⁶ cells, at least about 10⁷ cells, or more, which may be delivered systemically, by intratumoral injection, etc.

In some embodiments, expanded T cells are assayed for functional activity. Assays for functional activity include, without limitation, T cell cytotoxicity assays, IL-2 response, etc. as known in the art. Alternatively the T cells are assessed for the presence of markers indicative of activation, e.g. expression of CD3, CD25, CD69, CD137, CD107A, Granzyme B (GZMB), Perforin 1 (PRF1); etc. T cells can also be selected for an activated phenotype prior to administration.

Disclosed herein is a composition comprising an expanded population of activated immune cells. The composition can comprise tumor infiltrating lymphocytes derived from a culture comprising a patient derived organoid (PDO). The PDO can be grown in an air-liquid interface. The tumor infiltrating lymphocytes can express an mRNA or protein marker associated with immune activation. The marker associated with immune activation can be one or more of CD3, CD25, CD69, CD137, CD107A, Granzyme B (GZMB), or Perforin 1 (PRF1). The culture can comprise an agent that activates the tumor infiltrating lymphocytes. The agent can be an immune checkpoint inhibitor (ICI). The immune checkpoint inhibitor can be an anti-PD-1 antibody or an anti-CD47 antibody.

In some embodiments a therapeutic method is provided, the method comprising introducing into a recipient in need thereof of an expanded cell population as described above. The cell population is usually autologous or allogeneic with respect to the recipient.

As used herein, a “therapeutically effective amount” refers to that amount of the therapeutic agent sufficient to treat or manage a disease or disorder, e.g. at least about 10² cells/kg patient weight; 10³ cells/kg patient weight, 10⁴ cells/kg patient weight, 10⁵ cells/kg patient weight, 10⁶ cells/kg patient weight, 10⁷ cells/kg patient weight, 10⁸ cells/kg patient weight or more activated T cells.

As used herein, the term “dosing regimen” refers to a set of unit doses of cells (typically more than one) that are administered individually to a subject, typically separated by periods of time. In some embodiments, a dosing regimen comprises a plurality of doses each of which are separated from one another by a time period of the same length. In some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A subject in need of a therapy according to the herein described methods may be a subject in need of adoptive cell transfer (ACT) to treat the subject for cancer or for other disorders including infectious or autoimmune disease.

In one embodiment, a subject is treated using ACT employing an expanded cell population that has been activated and expanded by the methods disclosed herein. For example, cells may be collected from a subject, activated and expanded, and reintroduced into the subject as part of the ACT. The cells collected from the subject may be collected from any convenient and appropriate source for the ACT, including e.g., peripheral blood (e.g., the subject's peripheral blood), a biopsy (e.g., a tumor biopsy from the subject), and the like.

In some instances, the cells collected are tumor infiltrating lymphocytes (TILs), e.g., TILs collected from a tumor of a subject. In some instances, the cells collected are blood cells, e.g., NK cells collected from a subject's blood (e.g., a subject having cancer or a subject having an infection).

Following administration, an enhanced immune response may be manifest as an increase in the cytolytic response of T cells towards the target cells present in the recipient.

Combination Therapy. Treatment of a subject for a condition employing a composition and/or cells of the subject disclosure may, in some instances, be combined with one or more additional active agents. In some instances, useful additional active agents may include but are not limited to active agents for treating cancer. Alternatively, in some instances, a treatment method of the subject disclosure may exclude one or more additional, including any, active agents such that the treatment described is, e.g., the sole active composition (including cells) administered to the subject to treat the subject for the condition.

As summarized above, treatment may be combined with other active agents, including antibiotics, cytokines, and antiviral agents. Exemplary classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment.

Where treatment is directed to cancer, chemotherapeutic agents that can be administered in combination with the expanded cells include, without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, vumon, etc.

Targeted therapeutics that can be administered in combination with the expanded cells may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys (6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), Dabrafenib (Tafinlar); etc.

The expanded cells may be administered in combination with an immunomodulator, such as a cytokine, a lymphokine, a monokine, a stem cell growth factor, a lymphotoxin (LT), a hematopoietic factor, a colony stimulating factor (CSF), an interferon (IFN), a transforming growth factor (TGF), such as TGF-α or TGF-β, insulin-like growth factor (IGF), erythropoietin, thrombopoietin, a tumor necrosis factor (TNF) such as TNF-α or TNF-β, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, endostatin, and LT.

Tumor specific monoclonal antibodies that can be administered in combination with the expanded cells may include, without limitation, Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MEDI4736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin _V_3 (as used in the treatment of Tumor vasculature); Volociximab targeting Integrin _5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like. Cellular Compositions. Expanded cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Typically, compositions are prepared as injectables, either as liquid solutions or suspensions; solid forms suitable for solution in, or suspension in, liquid vehicles prior to injection can also be prepared. Proteins can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.Expanded T cells of the present disclosure may be used for other applications aside from immunotherapy. The methods of the present application may also be used for the identification of T cell receptor (TCR) clonotypes. In embodiments in which TCR clonotype is determined, T cells are expanded such that there are sufficient enough cells present in which a TCR clonotype may be determined from the T cell population within the PDO. Once TCR clonotype is determined, the TCR clonotype may then be used in the design of chimeric antigen receptor (CAR) T cell therapy. Other applications of the methods of the present disclosure include the use of PDOs as a means of screen the efficacy of immunotherapeutics or to determine the likelihood of a therapeutic agent being effective in the treatment of a specific individual wherein a sample from a tumor is taken and a PDO is generated from said tumor where the PDO is treated with a therapeutic agent and the efficacy of the agent is then evaluated.

The effect of an agent or cells, e.g. an immunotherapeutic agent, is determined by adding the agent or cells to the cells of the cultured explants as described herein, usually in conjunction with a control culture of cells lacking the agent or cells. The effect of the candidate agent or cell is then assessed by monitoring one or more output parameters. Parameters are quantifiable components of explants or the cells thereof, particularly components that can be accurately measured, in some instances in a high throughput system. For example, a parameter of the explant may be the growth, differentiation, survival, gene expression, proteome, phenotype with respect to markers etc. of the explant or the cells thereof, e.g. any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

### EXPERIMENTAL

### Examples

In TIL-based adoptive cell therapy (ACT), T cells are extracted from autologous tumors, followed by ex vivo reactivation and rapid expansion protocol (REP) to establish a bulk TIL product for an individual patient. TIL-based ACT has elicited durable and reproducible clinical benefits in treatment-naïve and/or refractory melanoma including anti-PD-1-resistant patients and cervical cancer. A major limitation is that the expanded TILs are not selected for tumor-reactivity, which dilutes overall efficacy and may impair the extension of this strategy from melanoma to tumor types with lesser neoantigen burdens. Ex vivo expanded TILs can be screened against autologous tumor cells but matched tumor cell lines are often not available during TIL REP and screening allogeneic tumor cell lines is not optimal. To improve ACT, efforts have pursued tumor antigens recognized by TIL TCRs including mutant KRAS. TIL neoantigen reactivity in vitro can be concentrated by PD-1 FACS and single cell cloning, but this is not a functional enrichment. However, CD8+ TILs expressing PD-1 expand, proliferate and kill autologous tumor cells and have distinct TCRαβ clonotypes versus PD-1-TILs and combining anti-PD-1 with PD-1+CD8+ TILs increases anti-tumor efficacy against B16-OVA.

Our studies indicate that PD-1/PD-L1 inhibitors reinvigorate and enrich tumor-reactive TILs in ALI tumor organoids.

Generation of human PDO cultures. Freshly acquired melanoma or cutaneous squamous cell cancer (cSCC) tumor tissue is routinely obtained by surgical excisional biopsy. Both melanoma and cutaneous squamous cell cancer exhibit high response rates to anti-PD-1 therapy (30-50%). The majority of the cSCC samples will be pre-treatment biopsies from locally advanced or metastatic tumors prior to anti-PD-1 monotherapy.

In vitro aPD-1 treatment and multiple timepoint sample collection. Anti-human PD-1 (cemiplimab or pembrolizumab to match patient treatment) or isotype control human IgG4 (10 μg/ml) is added immediately at ALI plating. PDOs are harvested for αPD-1 vs. control IgG4 at 3 different time points, between 1 h and 14 days, followed by (1) Annexin V/7-AAD FACS analysis of tumor cell death (melanoma: MCAM/CD146+CD31−CD45−; cSCC: EMA/CD227+), (2) FACS quantitation of CD3+, CD4+ and CD8+ subsets per 106 organoid cells and (3) αPD1-stimulated T cell activation and cytolysis by cell surface (CD25, CD69, CD137, CD107A) and intracellular FACS (GZMB, PRF1).

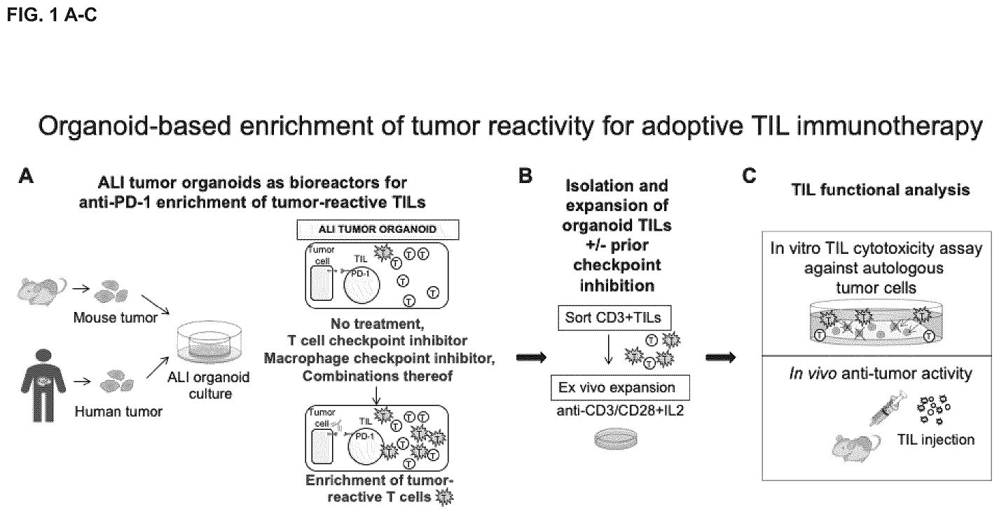

Organoid-based enrichment of tumor reactivity in adoptive TIL immunotherapies. ALI organoids were generated from B16-SIY melanoma tumors stably expressing SIYRYYGL peptide (SIY) implanted s.c. in syngeneic immunocompetent C57BI/6 mice (Cell, 2018). Like their human PDO counterparts, the B16-SIY organoids retain intrinsic TILs but are detectable by SIY tetramer FACS. Anti-PD-1 and anti-PD-L1 expand organoid SIY-reactive CD8+ T cells and induce Prf1, Gzmb, and Ifng mRNAs even after serial passage alongside tumor organoid killing. Thus, PD-1/PD-L1 inhibition enriches tumor-reactive TILs in ALI organoids. Further, anti-PD-1 IFNG/PRF1/GZMB TIL activation response of human PDOs positively correlates with PD-1 expression frequency on CD3+ TILs. To improve TIL-based immunotherapy human PDOs are used and mouse ALI tumor organoids as living bioreactors where in vitro checkpoint inhibition reinvigorates and enrich tumor-reactive TILs prior to ex vivo expansion and reinfusion in tumor models. These studies use (1) highly αPD-1-responsive mouse B16F10 MSI-high SIY ALI tumor organoids with their high anti-PD-1 response, overlaid with tumor antigen transduction to allow tetramer detection of tumor-reactive TILs and (2) clinically-relevant human melanoma PDOs (FIG. 1).

Ex vivo TIL expansion. In the traditional TIL protocol, pre-REP grows dissected tumor biopsies as fragments or single cells with IL-2; however, the tumor degenerates during this 11-day period. In the rapid expansion protocol (REP) phase, TILs are cultured for 2 weeks with αCD3, irradiated allogeneic PBMC-derived feeder cells, and IL-2 allowing expansion to ˜10¹¹ cells. Here, ALI tumor organoids substitute for pre-REP, utilizing organoids +/−αPD-1 instead of tumor fragments. Then, bulk organoid TILs (>10⁴) +/− prior to αPD-1 treatment are cultured with IL-2, αCD3 and irradiated allogeneic PBMC feeders, or anti-CD3/CD28 beads and IL-2 for expansion targeting 10⁸ cells for mouse studies; persistence of SIY pentamer-reactive TILs will be confirmed by FACS. In parallel, a 3rd condition will be processed by traditional pre-REP/REP from fresh tumor of the initial harvest.

TIL functional evaluation in vitro. REP-expanded TILs are functionally evaluated as follows:
Tumor cytotoxicity. T-cell mediated tumor organoid killing has been reported for co-cultured PBMCs and tumor organoids. This approach is used to test isolated or REP-expanded TILs for killing of B16F10-SIY MSI-high cells at varying E: T ratios.
TIL activation. REP-expanded TILs are stratified by SIY-pentamer FACS; both (+) and (−) fractions undergo FACS for activation (CD25, CD69, CD137) and cytolytic markers (extracellular CD107A; intracellular GZMB/PRF1), n=3 cultures. Enhanced tumor cytotoxicity and TIL activation +/−αPD-1 strongly indicate that organoid anti-PD-1 treatment enriches anti-tumor activity in REP TIL preparations. TILs extracted from αPD-1-treated B16-SIY organoids killed mouse B16-SIY melanoma grown in “epithelial-only” organoid culture; control splenic T cells were ineffective.

TIL functional evaluation in vivo. REP TIL preps with (1) control IgG, (2) aPD-1 organoid

treatment and (3) standard non-organoid REP are tested for anti-tumor activity. C57BL/6 mice receive total body irradiation (1000 rads) to deplete host T cells prior to adoptive TIL transfer. One day later, mice receive 107 TILs i.v., followed the next day by s.c. injection of 10⁶ B16F10-SIY MSI-high tumor cells (n=6 mice/group). Tumor size is measured by caliper measurement as mean +/−SE. Histology for tumor H&E, metastatic burden, apoptosis and CD3/CD4/CD8 TIL infiltration is performed. This is repeated in male and female mice. Caliper measurements of tumor growth are expressed as mean +/−SE. Tumor sizes of 1000±200 vs 300±200 mm3 in n=6 control Ig TIL mice and n=6 αPD-1 TIL mice yield >97% power and false-positive rate of 0.5%.

Generation and enrichment of tumor-reactive T cells in human PDOs. Murine ALI organoid findings are extended to similarly use human melanoma PDOs as a bioreactor platform for tumor-reactive TIL enrichment. Melanoma surgical biopsies are used to generate PDOs co-preserving immune and tumor cells and manifesting T cell activation upon 7 day anti-PD-1treatment. The melanoma PDOs are treated at plating with (1) control IgG4 or (2) pembrolizumab (10 μg/ml, 7 days). At day 7, the PDOs +/−αPD-1 undergo (A) FACS purification of a CD3⁺ TIL fraction for ex vivo expansion, and (B) MCAM⁺ CD45⁻ CD31⁻ tumor cells are re-seeded in submerged Matrigel for purely epithelial organoid growth for anti-tumor killing. αPD-1 TIL activation (CD25, CD69, CD137) and cytolytic markers (surface CD107A, intracellular GZMB and PRF1) are confirmed in an aliquot (5%) of CD3+ TILs as mean +/−SE % of triplicate cultures (c.f.). If checkpoint-regulated activity is confirmed, the remaining CD3+ TIL fraction (95%) proceeds to ex vivo expansion (REP).

Ex vivo REP expansion of human TILs. CD3+ TILs are isolated by FACS of +/−αPD-1-treated melanoma PDOs. The REP culture proceeds for 2 weeks with IL-2, αCD3 and irradiated allogeneic PBMC feeders. A 3rd condition will be processed by traditional pre-REP/REP from the initial biopsy.

In vitro functional evaluation of expanded human TILs. A significant advantage of organoids over traditional REP is the immediate availability of matched autologous tumor cells to confirm TIL anti-tumor activity. (a) REP TIL tumor cytotoxicity will be measured at varying E:T ratios by Annexin V/7-AAD FACS against pure epithelial tumor organoids in triplicate. (b) TIL activation is measured by activation (CD25, CD69, CD137) and cytolytic markers (surface CD107A, intracellular GZMB and PRF1), n=3. Enhanced tumor cytotoxicity and TIL activation in REP TIL preps by prior αPD-1 PDO treatment strongly support the use of PDOs as a bioreactor for enriching TIL anti-tumor activity.

In vivo functional evaluation for expanded human TILs. Each condition. i.e. (1) control IgG4, (2) anti-PD-1 and (3) traditional REP (n=6 mice/group) are evaluated for anti-tumor activity in the PDXv2.0 model, where tumor cells and autologous TILs are transplanted sequentially in hIL-2 NOG mice (lacking endogenous lymphocytes and bearing a CMV-driven human IL-2transgene (Taconic #13340)). The hIL-2 NOG mouse substantially promotes anti-tumor activity of ACT TIL therapies versus conventional NOG mice⁶⁰. 5×10⁵ autologous tumor cells from submerged Matrigel cultures containing tumor epithelium without immune cells are implanted s.c.

into hIL-2 NOG mice and randomized to each treatment group, n=6/group. The next day, 1×10⁶ autologous TILs from each REP are injected by tail vein and caliper measurements of tumor growth expressed as mean +/−SE. If organoid anti-PD-1 treatment results in REP inhibition of tumor size from 1000±200 to a new value of 300±200 mm³, n=6 mice/group yields >97% power with 0.5% false positive rate. Histology for tumor H&E, metastatic burden, apoptosis and CD3/CD4/CD8 TIL infiltration is performed. This is repeated in male and female mice.

### REFERENCES

1. Siegel, R. L., Miller, K. D. & Jemal, A. Cancer statistics, 2020. CA: a cancer journal for clinicians 70, 7-30 (2020).
2. Henley, S. J., et al. Annual report to the nation on the status of cancer, part I: National cancer statistics. Cancer 126, 2225-2249 (2020). PMC7299151
3. Flaherty, K. T., et al. Inhibition of mutated, activated BRAF in metastatic melanoma. N Engl J Med 363, 809-819 (2010). PMC3724529
4. Thommen, D. S. & Schumacher, T. N. T Cell Dysfunction in Cancer. Cancer Cell 33, 547-562 (2018).
5. Kerkar, S. P. & Restifo, N. P. Cellular constituents of immune escape within the tumor microenvironment. Cancer Res 72, 3125-3130 (2012).
6. Fridman, W. H., Pages, F., Sautes-Fridman, C. & Galon, J. The immune contexture in human tumours: impact on clinical outcome. Nat Rev Cancer 12, 298-306 (2012).
7. Chen, D. S. & Mellman, I. Oncology meets immunology: the cancer-immunity cycle. Immunity 39, 1-10 (2013).
8. Migden, M. R., et al. PD-1 Blockade with Cemiplimab in Advanced Cutaneous Squamous-Cell Carcinoma. N Engl J Med 379, 341-351 (2018).
9. Luksza, M., et al. A neoantigen fitness model predicts tumour response to checkpoint blockade immunotherapy. Nature 551, 517-520 (2017). 6137806
10. Zhong, S., et al. T-cell receptor affinity and avidity defines antitumor response and autoimmunity in T-cell immunotherapy. Proc Natl Acad Sci U S A 110, 6973-6978 (2013). 3637771
11. Goff, S. L., et al. Randomized, Prospective Evaluation Comparing Intensity of Lymphodepletion Before Adoptive Transfer of Tumor-Infiltrating Lymphocytes for Patients With Metastatic Melanoma. J Clin Oncol 34, 2389-2397 (2016). 4981979
12. Rosenberg, S. A., et al. Durable complete responses in heavily pretreated patients with metastatic melanoma using T-cell transfer immunotherapy. Clin Cancer Res 17, 4550-4557 (2011). 3131487
13. Sarnaik, A., et al. Safety and efficacy of cryopreserved autologous tumor infiltrating lymphocyte therapy (LN-144, lifileucel) in advanced metastatic melanoma patients following progression on checkpoint inhibitors. in SITC Annual Meeting (Washington, DC, USA, 2018).
14. Jazaeri, A., et al. A Phase 2, Multicenter Study to Evaluate the Efficacy and Safety Using Autologous Tumor Infiltrating Lymphocytes (LN-145) in Patients with Recurrent, Metastatic, or Persistent Cervical Carcinoma. in ASCO Annual Meeting (CHICAGO, IL, USA, 2018).
15. Rizvi, N. A., et al. Cancer immunology. Mutational landscape determines sensitivity to PD-1 blockade in non-small cell lung cancer. Science 348, 124-128 (2015).
16. Donnem, T., et al. Strategies for clinical implementation of TNM-Immunoscore in resected nonsmall-cell lung cancer. Ann Oncol 27, 225-232 (2016).
17. Mlecnik, B., et al. Histopathologic-based prognostic factors of colorectal cancers are associated with the state of the local immune reaction. J Clin Oncol 29, 610-618 (2011).
18. Deschoolmeester, V., et al. Tumor infiltrating lymphocytes: an intriguing player in the survival of colorectal cancer patients. BMC immunology 11, 19 (2010). 2864219
19. Feder-Mengus, C., Ghosh, S., Reschner, A., Martin, I. & Spagnoli, G. C. New dimensions in tumor immunology: what does 3D culture reveal? Trends in molecular medicine 14, 333-340 (2008).
20. Hirt, C., et al. “In vitro” 3D models of tumor-immune system interaction. Advanced drug delivery reviews 79-80, 145-154 (2014).
21. Boj, S. F., et al. Organoid models of human and mouse ductal pancreatic cancer. Cell 160, 324-338 (2015).
22. Huang, L., et al. Ductal pancreatic cancer modeling and drug screening using human pluripotent stem cell-and patient-derived tumor organoids. Nat Med 21, 1364-1371 (2015). PMC4753163
23. Ohlund, D., et al. Distinct populations of inflammatory fibroblasts and myofibroblasts in pancreatic cancer. J Exp Med (2017).
24. Dijkstra, K. K., et al. Generation of Tumor-Reactive T Cells by Co-culture of Peripheral Blood Lymphocytes and Tumor Organoids. Cell (2018).
25. Chen, J., et al. Single-cell transcriptome analysis identifies distinct cell types and intercellular niche signaling in a primary gastric organoid model. bioRxiv 190132 .
26. Finnberg, N. K., et al. Application of 3D tumoroid systems to define immune and cytotoxic therapeutic responses based on tumoroid and tissue slice culture molecular signatures. Oncotarget 8, 66747-66757 (2017). PMC5620133
27. Jenkins, R. W., et al. Ex Vivo Profiling of PD-1 Blockade Using Organotypic Tumor Spheroids. Cancer Discov 8, 196-215 (2018). PMC5809290
28. Deng, J., et al. CDK4/6 Inhibition Augments Antitumor Immunity by Enhancing T-cell Activation. Cancer Discov 8, 216-233 (2018). PMC5809273
29. Neal, J. T., et al. Organoid Modeling of the Tumor Immune Microenvironment. Cell 175, 1972-1988 e1916 (2018).
30. Ootani, A., et al. Sustained in vitro intestinal epithelial culture within a Wnt-dependent stem cell niche. Nat Med 15, 701-706 (2009).
31. Li, X., et al. Oncogenic transformation of diverse gastrointestinal tissues in primary organoid culture. Nat Med 20, 769-777 (2014). PMC4087144
32. Topalian, S. L., et al. Safety, activity, and immune correlates of anti-PD-1 antibody in cancer. N Engl J Med 366, 2443-2454 (2012). 3544539
33. Borghaei, H., et al. Nivolumab versus Docetaxel in Advanced Nonsquamous Non-Small-Cell Lung Cancer. N Engl J Med 373, 1627-1639 (2015).
34. Brahmer, J., et al. Nivolumab versus Docetaxel in Advanced Squamous-Cell Non-Small-Cell Lung Cancer. N Engl J Med 373, 123-135 (2015). PMC4681400
35. Garon, E. B., et al. Pembrolizumab for the treatment of non-small-cell lung cancer. N Engl J Med 372, 2018-2028 (2015).
36. Herbst, R. S., et al. Pembrolizumab versus docetaxel for previously treated, PD-L1-positive, advanced non-small-cell lung cancer (KEYNOTE-010): a randomised controlled trial. Lancet 387, 1540-1550 (2016).
37. Kline, J., Zhang, L., Battaglia, L., Cohen, K. S. & Gajewski, T. F. Cellular and molecular requirements for rejection of B16 melanoma in the setting of regulatory T cell depletion and homeostatic proliferation. J Immunol 188, 2630-2642 (2012). PMC3294164
38. Sockolosky, J. T., et al. Durable antitumor responses to CD47 blockade require adaptive immune stimulation. Proc Natl Acad Sci U S A 113, E2646-2654 (2016). PMC4868409
39. Sockolosky, J. T., et al. Selective targeting of engineered T cells using orthogonal IL-2 cytokine-receptor complexes. Science 359, 1037-1042 (2018). PMC5947856
40. Gordon, S. R., et al. PD-1 expression by tumour-associated macrophages inhibits phagocytosis and tumour immunity. Nature 545, 495-499 (2017).
41. Chen, P. L., et al. Analysis of Immune Signatures in Longitudinal Tumor Samples Yields Insight into Biomarkers of Response and Mechanisms of Resistance to Immune Checkpoint Blockade. Cancer Discov 6, 827-837 (2016). PMC5082984
42. Mandal, R., et al. Genetic diversity of tumors with mismatch repair deficiency influences anti-PD-1 immunotherapy response. Science 364, 485-491 (2019). PMC6685207
43. Gros, A., et al. PD-1 identifies the patient-specific CD8 (+) tumor-reactive repertoire infiltrating human tumors. J Clin Invest 124, 2246-2259 (2014). PMC4001555
44. Li, H., et al. Dysfunctional CD8 T Cells Form a Proliferative, Dynamically Regulated Compartment within Human Melanoma. Cell (2018).
45. Butler, A., Hoffman, P., Smibert, P., Papalexi, E. & Satija, R. Integrating single-cell transcriptomic data across different conditions, technologies, and species. Nat Biotechnol 36, 411-420 (2018). PMC6700744
46. Aoki, K. F. & Kanehisa, M. Using the KEGG database resource. Curr Protoc Bioinformatics Chapter 1, Unit 1 12 (2005).
47. Browaeys, R., Saelens, W. & Saeys, Y. NicheNet: modeling intercellular communication by linking ligands to target genes. Nat Methods (2019).
48. Zou, H. & Zhang, H. H. On the Adaptive Elastic-Net with a Diverging Number of Parameters. Ann Stat 37, 1733-1751 (2009). PMC2864037
49. Shugay, M., et al. VDJtools: Unifying Post-analysis of T Cell Receptor Repertoires. PLOS Comput Biol 11, e1004503 (2015). PMC4659587
50. Glanville, J., et al. Identifying specificity groups in the T cell receptor repertoire. Nature 547, 94-98 (2017). 5794212
51. Hou, Y., et al. Non-canonical NF-KB Antagonizes STING Sensor-Mediated DNA Sensing in Radiotherapy. Immunity 49, 490-503 e494 (2018). PMC6775781
52. Dudley, M. E., Wunderlich, J. R., Shelton, T. E., Even, J. & Rosenberg, S. A. Generation of tumor-infiltrating lymphocyte cultures for use in adoptive transfer therapy for melanoma patients. Journal of immunotherapy (Hagerstown, Md.: 1997) 26, 332-342 (2003). PMC2305721
53. Rosenberg, S. A. & Restifo, N. P. Adoptive cell transfer as personalized immunotherapy for human cancer. Science 348, 62-68 (2015). PMC6295668
54. Svane, I. M. & Verdegaal, E. M. Achievements and challenges of adoptive T cell therapy with tumor-infiltrating or blood-derived lymphocytes for metastatic melanoma: what is needed to achieve standard of care? Cancer immunology, immunotherapy: CII 63, 1081-1091 (2014).
55. Yossef, R., et al. Enhanced detection of neoantigen-reactive T cells targeting unique and shared oncogenes for personalized cancer immunotherapy. JCI Insight 3 (2018). PMC6237474
56. Garber, K. Driving T-cell immunotherapy to solid tumors. Nat Biotechnol 36, 215-219(2018).
57. Tran, E., Robbins, P. F. & Rosenberg, S. A. ‘Final common pathway’ of human cancer immunotherapy: targeting random somatic mutations. Nat Immunol 18, 255-262 (2017). PMC6295671
58. Tran, E., et al. T-Cell Transfer Therapy Targeting Mutant KRAS in Cancer. N Engl J Med 375, 2255-2262 (2016). PMC5178827
59. Fernandez-Poma, S. M., et al. Expansion of Tumor-Infiltrating CD8(+) T cells Expressing PD-1 Improves the Efficacy of Adoptive T-cell Therapy. Cancer Res 77, 3672-3684 (2017).
60. Jespersen, H., et al. Clinical responses to adoptive T-cell transfer can be modeled in an autologous immune-humanized mouse model. Nat Commun 8, 707 (2017). PMC5617838
61. Dijkstra, K. K., et al. Generation of Tumor-Reactive T Cells by Co-culture of Peripheral Blood Lymphocytes and Tumor Organoids. Cell 174, 1586-1598.e1512 (2018).
62. Simoni, Y., et al. Bystander CD8(+) T cells are abundant and phenotypically distinct in human tumour infiltrates. Nature 557, 575-579 (2018).
63. Duhen, T., et al. Co-expression of CD39 and CD103 identifies tumor-reactive CD8 T cells in human solid tumors. Nat Commun 9, 2724 (2018). PMC6045647
64. Canale, F. P., et al. CD39 Expression Defines Cell Exhaustion in Tumor-Infiltrating CD8(+) T Cells. Cancer Res 78, 115-128 (2018).

What is claimed is:

1. A method for the treatment of cancer in an individual, the method comprising:
a) culturing a tumor tissue sample in a gel with an air-liquid interface to provide a patient specific organoid (PDO) in the presence of immune cells;
b) contacting the PDO with or without an agent that activates the immune cells for a period of time sufficient to modulate immune cell activity;
c) culturing the activated immune cells to generate an expanded population of activated immune cells; and
d) administering an effective dose of the expanded population of activated immune cells to the individual in which the tumor tissue sample was obtained from.

2. The method of claim 1, wherein the immune cells comprise T cells.

3. The method of claim 1 or claim 2, wherein the immune cells are isolated from the individual.

4. The method of any of claims 1-3, wherein the immune cells are tumor infiltrating lymphocytes.

5. The method of any of claims 1-4, wherein the immune cells comprise one or more of B cells, NK cells, dendritic cells, macrophages, myeloid derived suppressor cells and T cells.

6. The method of any of claims 1-5, wherein the agent that activates immune cells is an immune checkpoint inhibitor (ICI) modulating T cells, macrophages or the like.

7. The method of any of claims 1-5, without treatment with an agent that activates immune cells such as an immune checkpoint inhibitor (ICI) modulating T cells, macrophages or the like.

8. The method of claim 6, wherein the ICI is an anti-PD-1 antibody.

9. The method of claim 6, wherein the ICI is an anti-CD47 antibody.

10. The method of any of claims 1-9, wherein activation of immune cells is determined by measuring expression of an mRNA or protein marker associated with immune activation.

11. The method of claim 10 wherein the marker comprises one or more of CD3, CD25, CD69, CD137, CD107A, Granzyme B (GZMB), or Perforin 1 (PRF1).

12. The method of any of claims 1-11, wherein the expanded population of immune cells are selected to provide a population enriched for activated cells.

13. The method of claim 12, wherein the activated immune cell is isolated using flow cytometry.

14. The method of any of claims 1-11, wherein the cancer is selected from clear cell renal cell carcinoma, ampullary carcinoma, cutaneous SCC, melanoma, lung adenocarcinoma, non-small lung cell cancer, gastrointestinal cancer, pancreatic cancer, colorectal cancer, hepatocellular carcinoma, cholangiocarcinoma, combined hepatocellular cholangiocarcinoma, Barrett's oesophagus cancer, intestinal carcinoma, prostate cancer, bladder cancer, breast cancer, glioblastoma, or cutaneous squamous cell carcinoma.

15. The method of claim 14, wherein the cancer is renal cell carcinoma or cutaneous squamous cell carcinoma.

16. The method of any of claims 1-15, wherein the immune cell is cultured for 7 days with the agent that activates the immune cell.

17. The method of any of claims 1-16, wherein step (c) comprises a rapid expansion protocol (REP) of culturing activated immune cells with interlukin-2 (IL-2), an anti-CD3 antibody and an irradiated allogenic population peripheral blood mononuclear cells (PBMCs).

18. The method of claim 17, wherein the REP comprising culturing cells for 14 days.

19. The method of any of claims 1-18, further comprising administering a second agent in step (b) in addition to the first agent that activates immune cells.

20. The method of claim 1, further comprising contacting a PDO in absence of stromal and immune cells with the expanded population of activated immune cells to determine tumor cell killing by the expanded immune cell population.

21. A composition, the composition comprising the expanded population of activated immune cells of any of claims 1-20.

22. A composition, the composition comprising tumor infiltrating lymphocytes wherein the tumor infiltrating lymphocytes are derived from a culture comprising a patient derived organoid.

23. The composition of claim 22, wherein the tumor infiltrating lymphocytes expresses an mRNA or protein marker associated with immune activation.

24. The composition of claim 23, wherein the marker is one or more of CD3, CD25, CD69, CD137, CD107A, Granzyme B (GZMB), or Perforin 1 (PRF1).

25. The composition of any of claims 22-24, wherein the culture further comprises an agent that activates the tumor infiltrating lymphocytes.

26. The composition of claim 25, wherein the agent is an immune checkpoint inhibitor (ICI).

27. The composition of claim 26, wherein the immune checkpoint inhibitor is an anti-PD-1 antibody.

28. The composition of claim 27, wherein the immune checkpoint inhibitor is an anti-CD47 antibody.
